(19) United States
(12) Patent Application Publication
(10) Pub. No.: US 2007/0213689 A1
(43) Pub. Date: Sep. 13, 2007

(54) Deflectable tip infusion guidewire

(76) Inventors: David D. Grewe, Glen Gardner, NJ (US); Hikmat Hojeibane, Princeton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/590,536

(22) Filed: Oct. 31, 2006

Related U.S. Application Data
(63) Continuation-in-part of application No. 10/691,823, filed on Oct. 23, 2003.
Application No. 10/691,823 is a continuation-in-part of application No. 10/224,168, filed on Aug. 20, 2002, now Pat. No. 7,128,718.
(60) Provisional application No. 60/366,739, filed on Mar. 22, 2002.

(51) Int. Cl. A61M 25/08

(52) U.S. Cl. 604/526

(57) ABSTRACT

A steerable infusion guidewire having a deflectable distal tip which comprises a longitudinal hypotube and an interlocking spring coil which carry an infusion tube which is attached to the distal end of the hypotube and also includes a longitudinally movable deflection member which is attached to the distal end of the spring coil and a tip retaining member which extends from the distal end of the hypotube to the distal end of the spring coil for providing very precise deflection of the distal tip of the guidewire.

### CROSS-REFERENCE TO RELATED APPLICATIONS(S)

This patent application is a continuation-in-part of U.S. patent application Ser. No. 10/691,823 (Attorney Docket No. CRD1061USACIP1), filed on Oct. 23, 2003, entitled, “Guidewire With Deflectable Tip Having Improved Torque Characteristics,” which is a continuation-in-part of U.S. patent application Ser. No. 10/224,168 (Attorney Docket No. CRD1061USNP), filed on Aug. 20, 2002, entitled, “Guidewire With Deflectable Tip,” now issued as U.S. Pat. No. 7,128,718, which is a nonprovisional patent application of U.S. patent application Ser. No. 60/366,739 (Attorney Docket No. CRD1035USPSP), filed on Mar. 22, 2002, entitled, “Deflection Wire Concept.”

### BACKGROUND OF THE INVENTION

1. Field of the Invention

The present invention relates to a steerable infusion guidewire having improved torque characteristics, and more particularly to a bi-directional steerable infusion guidewire having a tip which may be very precisely “steered,” or deflected. The infusion guidewire is particularly suitable for use in conjunction with the insertion of a catheter into a vessel of the body, or alternatively, the infusion guidewire may be used by itself to open obstructions within a vessel or for infusing a therapeutic fluid for removing obstructions within a vessel.

2. Description of the Prior Art

For many years guidewires have included a core wire with the distal end being tapered and with a coil spring mounted on the tapered distal end. These guidewires have been used to facilitate the insertion of a catheter into a vessel of the body. Generally, the guidewire is inserted into a vessel, a catheter is inserted over the guidewire and the catheter is then moved through the vessel until the distal end of the catheter is positioned at a desired location. The guidewire is then retracted from the catheter and the catheter is left in the vessel. Alternatively, the guidewire may be first inserted into the catheter with the distal portion of the guidewire extending beyond the distal end of the catheter. This assembly is then inserted into a vessel with the distal tip of the guidewire being used to facilitate movement of the guidewire and catheter through the vessel. Again, when the distal tip of the catheter has been placed in a desired location, the guidewire may be retracted thereby leaving the catheter in place within the vessel.

Another common application for guidewires is that of using the distal tip of the guidewire for removing an obstruction within a vessel. Often times this procedure is accomplished by inserting the guidewire within a vessel, moving the distal tip of the guidewire into contact with the obstruction and then very gently tapping the distal tip of the guidewire against the obstruction until the guidewire passes through the obstruction. Alternatively, various types of devices may be placed on the distal end of a guidewire for actively opening an obstruction within the vessel. Examples of such devices which may be placed on the end of the guidewires in order to open an obstruction are disclosed in the following Robert C. Stevens U.S. Pat. Nos. 5,116,350; 5,078,722; 4,936,845; 4,923,462; and, 4,854,325.

While most guidewires used today do not include a mechanism for deflecting or steering the tip of the guidewire, it is very desirable to provide tip steering in order to facilitate movement of the guidewire through the tortuous vessels of the body. There are many patents directed toward different mechanisms for deflecting the distal tip of a guidewire in order to steer the guidewire. Examples of such guidewires are disclosed in the following patents: U.S. Pat. No. 4,815,478 to Maurice Buchbinder, et al., U.S. Pat. No. 4,813,434 to Maurice Buchbinder, et al., U.S. Pat. No. 5,037,391 to Julius G. Hammerslag, et al., U.S. Pat. No. 5,203,772 to Gary R. Hammerslag, et al., U.S. Pat. No. 6,146,338 to Kenneth C. Gardeski, et al., U.S. Pat. No. 6,126,649 to Robert A. VanTassel, et al., U.S. Pat. No. 6,059,739 to James C. Baumann and U.S. Pat. No. 5,372,587 to Julius G. Hammerslag, et al. U.S. Pat. No. 4,940,062 to Hilary J. Hampton, et al., discloses a balloon catheter having a steerable tip section. All of the above-identified patents are incorporated herein by reference.

Still further, when a guidewire is passed through or around an obstruction it may then be desirable to infuse a therapeutic fluid into the vessel at a location beyond the obstruction. Examples of fluids which have in the past been infused into blood vessels are Sirolimus (for treating unstable aneurysm) contrast media (to illuminate the lumen of a vessel with X-rays), chemotherapeutic liquids (treatment of cells).

While each of these guidewires have some degree of steerability, there is a need to have an infusion guidewire with very precise steering and a very small diameter which is suitable for the purposes described above. More particularly, there is an important need for a very small diameter infusion guidewire having improved torque characteristics which includes a distal tip which may be deflected very precisely in either of two directions to enhance steerability.

### SUMMARY OF THE INVENTION

In accordance with one aspect of the present invention, there is provided a very small diameter steerable infusion guidewire having a deflectable tip which includes an elongated flexible tubing, a flexible helical coil attached to the distal portion of the flexible tubing, an elongated deflection member which is slidably disposed within the tubing and within the helical coil. The flexible helical coil is formed from an elongated member having a rectangular, or square cross section, and having continuous undulations wherein the undulations of adjacent turns interlock with each other, i.e., peak undulation of one turn interlocking with valley undulation of adjacent turn, to thereby enhance the rotational rigidity, referred to as torque characteristic, of the coil. The proximal portion of the deflection member is of a cylindrical configuration and the distal portion is tapered to form a deflection ribbon. Alternatively, the deflection member may take the form of a proximal cylindrical wire which is attached at its distal end to a deflection ribbon. In addition, a retaining ribbon is attached to the distal end of the flexible tubing and is oriented to extend in a plane which is generally parallel to the plane of the ribbon portion of the deflection member. An attachment member which may take the form of a rounded bead, preferably formed from epoxy, is bonded to the distal end of the helical coil, the distal end of the deflection ribbon and the distal end of the retaining ribbon so that longitudinal movement of the deflection member causes the distal end of the helical coil to be deflected. An infusion tube extends through the control passage of the device and extends from the proximal end of the device to a position which corresponds to the distal end of the elongated flexible tubing to create a passage for a therapeutic fluid to be infused through the device and then through spaces between the turns of the helical coil. Alternatively, the infusion tube may extend to the distal end of the device and pass through the distal rounded bead in order to infuse fluid through the distal tip of the device. With the enhanced rotational rigidity of the coil portion or the infusion guidewire, the entire infusion guidewire has enhanced rotational rigidity.

In accordance with another aspect of the present invention, the continuous undulations take the form of a sinusoidal wave, or alternatively a square sinusoidal wave, having positive and negative peaks and in which the positive peaks of adjacent turns of coils engage negative peaks, or valleys, of adjacent turns.

In accordance with another aspect of the present invention, the retaining ribbon and the deflection ribbon are preferably pre-shaped into a curved configuration to thereby cause the flexible helical coil to be biased into a normally curved shape.

In accordance with a further aspect of the present invention, the distal portion of the deflection ribbon engages the attachment member, or rounded bead, at a location offset from the center of the attachment member, and the distal portion of the retaining ribbon engages the attachment member at a location offset from the center of the attachment member. Preferably, the retaining ribbon engages the attachment member at a location offset from the center portion of the attachment member in the opposite direction from the offset location of the deflection ribbon.

In accordance with still another aspect of the present invention, the deflection ribbon and the retaining ribbon are connected to each other within the attachment member. Preferably these two elements are formed as a single unitary element. In a preferred embodiment of the invention the cylindrical deflection member is flattened to form the deflection ribbon and is further flattened at its distal end to form the retaining ribbon. The retaining ribbon is bent 180 degrees with respect to the deflection ribbon to form a generally U-shaped bend to thereby establish a predetermined spacing between the ribbons and to also cause these ribbons to remain parallel to each other.

### BRIEF DESCRIPTION OF THE DRAWINGS

FIG. 1 is an enlarged elevational view of a balloon on an infusion guidewire having a deflectable tip and control handle in accordance with the one aspect of the present invention;

FIGS. 2A and 2B are enlarged elevational sectional views showing two embodiments of the distal end of the infusion guidewire in its normal pre-shaped position;

FIGS. 3 and 4 are sectional views showing the steerable infusion guidewire deflected from its normal position to opposite extremes of deflection.

### DESCRIPTION OF THE PREFERRED EMBODIMENT

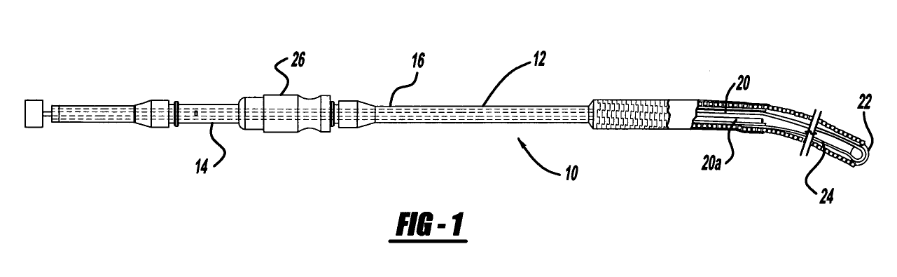

FIG. 1 generally illustrates a steerable infusion guidewire system 10 which embodies the present invention and comprises a steerable infusion guidewire 12 coupled to a control handle 14. More particularly, the steerable infusion guidewire comprises an elongated hypotube 16, a helical coil 18 attached to and extending from the distal end of the hypotube 16. The helical coil 18 is of a rectangular or square cross-sectional configuration and is preferably formed from platinum tungsten with the proximal turns being wound such that adjacent turns of the proximal portion are in contact, or loosely interlocked with each other.

While the preferred embodiment of the present invention includes the helical coil 18, this element may take the form of any flexible rectangular or square cross-sectional member, such as for example a thin square metallic tube with or without portions of the tube removed, for example laser cutting, so as to form a very flexible cylindrical or square member. An elongated deflection member 20 extends from the proximal end of the control handle through the hypotube 16 and through the helical coil 18, and is connected into an attachment member, or rounded bead 22, which is disposed at the distal tip of the helical coil 18. In addition, a retaining ribbon 24 is connected to the distal end of the hypotube 16 and is also connected to the rounded bead 22.

The control handle 14 generally comprises a slidable control knob 26 which may be moved longitudinally with respect to the control handle. The control handle 14 is coupled to the deflection member 20. As will be discussed in more detail, the longitudinal movement of the slidable control knob 26 causes deflection of the distal tip of the infusion guidewire in either an upward or downward direction.

A fluid infusion tube 20a extends from the proximal end of the infusion guidewire through the hypotube 16 to a position corresponding to the location where the helical coil 18 joins the elongated hypotube 16.

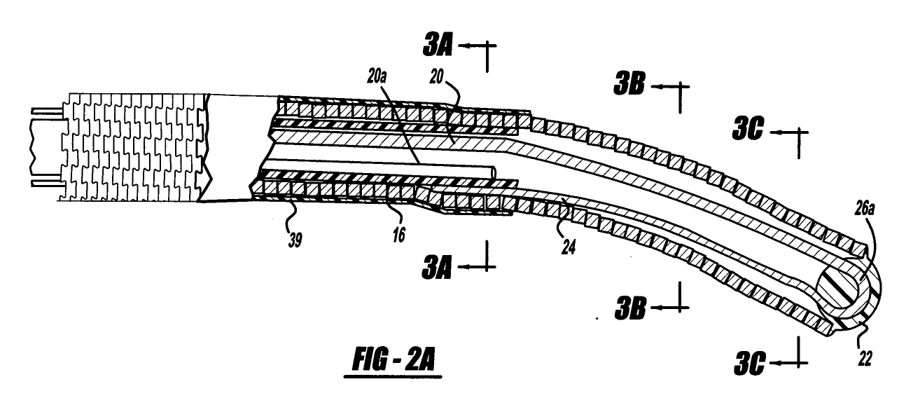

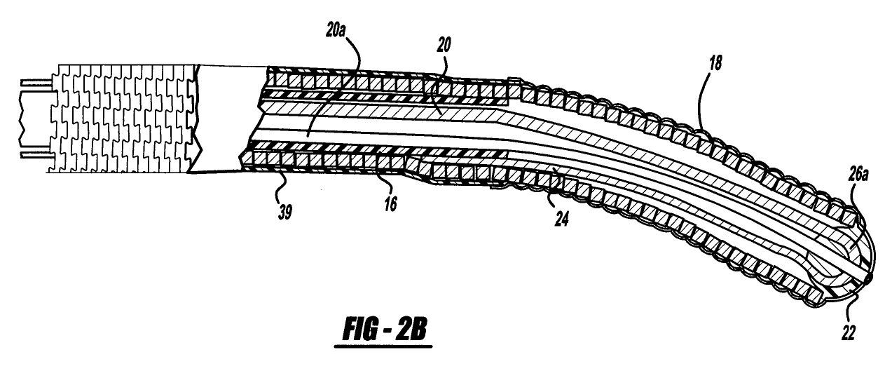

FIGS. 2A and 2B illustrate in more detail two embodiments of the distal portion of the infusion guidewire 12. More particularly, the proximal end of the helical coil 18 is bonded, preferably by use of an epoxy, to the outer surface near the distal end of the hypotube 16. The elongated deflection member 20 takes the form of a small diameter cylindrical deflection member 20 having an intermediate portion which is flattened to form a thin ribbon having a thickness of approximately 0.002 inches. The distal end of the cylindrical deflection member 20 is further flattened to a thickness of approximately 0.0015 inches and is bent back 180 degrees to form a U-shaped bend 26a between the deflection member 20 and the retaining ribbon 24. The proximal end of the retaining ribbon 24 is bonded, preferably by use of epoxy, to the outer surface of the distal end of the hypotube 16. The retaining ribbon 24 is aligned in a plane parallel to the plane of the deflection member 20 and the U-shaped portion between the ribbons is encapsulated by the attachment member which preferably takes the form of a rounded epoxy bead 22 bonded to the distal tip of the helical coil 18.

As may be appreciated, with this unitary construction of the deflection member 20 and the retaining ribbon 24, these members remain aligned so that both lie in planes parallel to each other. In addition, the U-shaped bend portion when encapsulated into the rounded bead 22 causes the retaining ribbon and deflection ribbon to be properly spaced with respect to each other.

As illustrated in FIGS. 2A and 2B, the retaining ribbon 24 is preferably attached to the rounded bead 22 at a position offset from the center of the bead in the same direction that the retaining ribbon 24 is offset from the longitudinal axis of the steerable infusion guidewire 12. In addition, the deflection member 20 is attached to the bead at a position offset from the center of the bead in an opposite direction from the offset of the retaining ribbon 24.

Also, the deflection member 20 and the retaining ribbon 24 are pre-shaped into an arcuate, or curved, configuration to thereby maintain the helical coil 18 in a normally curved configuration. The distal sections of the deflection member 20 and the retaining ribbon 24 are pre-shaped such that the distal tip of the infusion guidewire curves away from the longitudinal axis of the infusion guidewire in a direction toward that side of the infusion guidewire containing the retaining ribbon 24.

The helical coil 18 is formed as an elongated member having a rectangular, or square, cross-sectional configuration and wound in a helical configuration. In addition, the elongated member is preferably formed with re-occurring steps, or step undulations, which when wound into a helical configuration so that adjacent turns to loosely interlock thereby preventing movement between adjacent turns. Such interlocking turns enhance the rotational rigidity or “torqueability” of the coil such that when the proximal end of the coil is rotated 180 degrees, the distal end of the coil will rotate approximately 180 degrees. Accordingly, the distal end of the coil more nearly tracks, rotationally, the proximal end of the coil thereby significantly improving the “tortional” characteristics of the coil. By improving the “tortional” characteristics of the coil, the overall “tortional” characteristics of the infusion guidewire are significantly improved.

As opposed to winding an elongated member to form the helical coil 18, a preferred method of forming the helical coil is by laser cutting the coil from a single thin-walled tube of an alloy in the undulations locking, stepped configuration. Such laser cutting provides a coil with precise mating surfaces to assure proper interlocking between adjacent turns of the helical coil.

In the embodiment illustrated in FIG. 2A, the infusion tub terminates at a location in the vicinity of the position where the helical coil 18 joins the hypotube 16 so that fluid passing through the infusion tube is infused through the spacing between turns of the helical coil 18. Alternatively, in the embodiment illustrated in FIG. 2B, the infusion tube 20a extends distally through the rounded bead 20 for infusing fluid distally of the infusion guidewire.

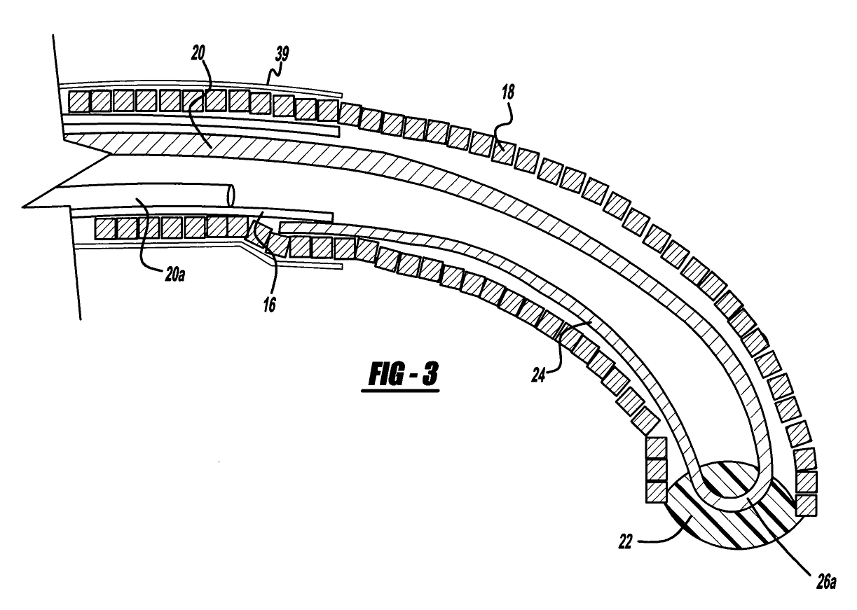

In operation, as previously described, the distal tip of the steerable infusion guidewire 12 is normally biased into a downwardly curved position as illustrated in FIGS. 2A and 2B because of the curve of the pre-shaped deflection member 20 and the retaining ribbon 24. When the slidable control knob 26 is moved distally as shown in FIG. 3, the deflection member 20 will be moved distally thereby causing the distal end of the deflection member to move in a distal direction. As the deflection member is moved distally, a pushing force is applied to the top portion of the rounded bead 22. The retaining ribbon 24 is attached to the lower portion of the bead 22 to thereby maintain the bead at a fixed distance from the distal end of the hypotube 16. As the deflection member 20 is moved to the right, the tip of the infusion guidewire is caused to deflect downwardly to a maximum deflected position.

Since the deflection member 20 and the retaining ribbon 24 are pre-shaped prior to any activation of the steerable infusion guidewire, the amount of force required to deflect the infusion guidewire in this direction is very small thereby preventing buckling of the deflection member 20 as it is pushed distally. As the deflection member 20 is moved distally, the upper turns of the helical coil become slightly stretched and the lower turns of the coil become slightly compressed. The deflection member 20 has a diameter of about 0.0065 inches and the deflection ribbon has a thickness of about 0.002 inches to thereby provide sufficient stiffness to prevent the buckling of these elements when the deflection member 20 is pushed distally. This construction also provides sufficient stiffness to transmit the necessary force from the proximal end to the distal end of the infusion guidewire.

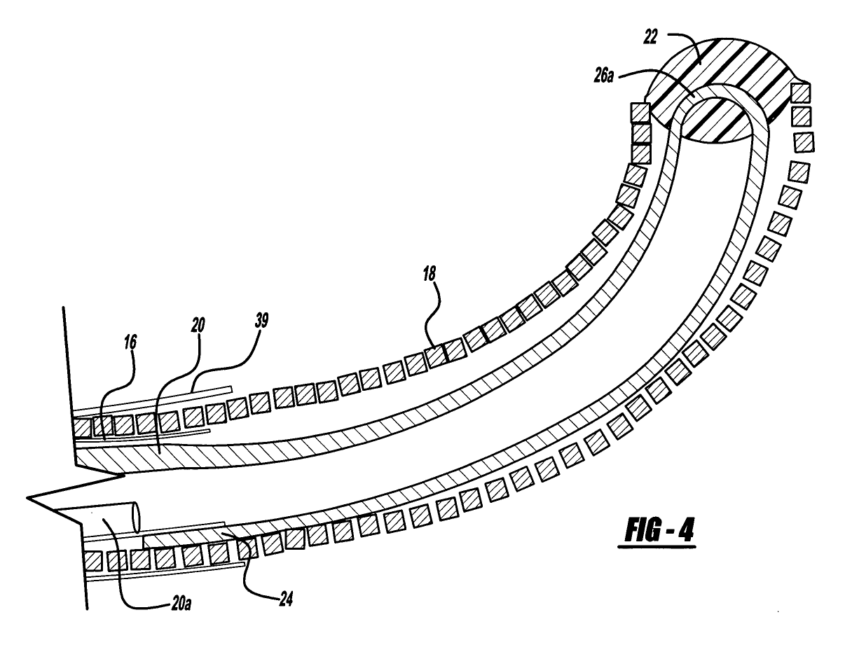

When the slidable control knob 26 is moved in a proximal direction as shown in FIG. 4, the deflection member 20 will be pulled to the left to thereby cause the deflection member 20 to pull on the top portion of the bead 22. Since again the retaining ribbon 24 causes the lower portion of the bead to remain at a fixed distance from the distal end of the hypotube 16, the tip of the infusion guidewire 12 is caused to bend in an upward direction to a maximum deflection as shown in FIG. 4. Since the deflection member 20 is in tension when it is pulled, there is no concern for buckling of the deflection member. As the deflection member 20 is moved proximally, the upper coil turns become slightly compressed and the lower coil turns become somewhat stretched.

In the embodiment of the device illustrated in FIG. 2A a thereapeutic fluid may be infused through turns of the coil. In the embodiment of the device illustrated in FIG. 2B the fluid may be infused out of the distal tip of the device.

As previously discussed, when the proximal end of the infusion guidewire 12 is rotated by a physician to “steer” the distal end of the infusion guidewire, with the interlocking turns of adjacent coils of the helical coil 18, the distal tip will rotate on a one-to-one basis with respect to the proximal end of the hypotube 16. In other words, there is no “play” or “lag” between rotation of the proximal end and the distal end of the infusion guidewire.

In a preferred embodiment of the present invention, the elongated deflection member 20 and the retaining ribbon 24 are constructed of nitinol, but these elements may be formed from other flexible materials including polymers. The helical coil 18 preferably formed by laser cutting as previously discussed, is constructed from an alloy comprised of about 92 percent platinum and 8 percent tungsten, but this element may also be constructed from numerous other materials. It is desirable that the coil exhibit the characteristic of being radiopaque to X-rays to assist in the positioning of the distal tip of the steerable infusion guidewire 12. The deflection member 20 is formed from a single cylindrical nitinol wire of about 0.0065 inches in diameter having an intermediate portion which is flattened to form the distal section of the deflection member 20 with a thickness of about 0.002 inches, and a distal portion which is flattened to form the retaining ribbon 24 with a thickness of about 0.0015 inches. The retaining ribbon 24 is bent back 180 degrees to form a generally U-shaped bend, which is subsequently encapsulated within the rounded bead 22. The rounded bead 22 is preferably formed with epoxy, but may be formed with soldering or by welding.

It has been found that the addition of graphite between the deflection member 20 and the inner lumen of the hypotube 16 provides lubrication. Other lubricants, such as Teflon or MDX may be used for this purpose. The helical coil 18 is preferably coated with an elastomeric polymer on its distal end to act as a sealant preventing the entry of blood and contrast media into the infusion guidewire and a fluorinated polymer 39, such as Teflon, on its proximal end for lubrication purposes.

It may be seen that the infusion guidewire as disclosed may be very easily and very precisely rotated and then deflected in either of two directions for very precise steering of the infusion guidewire through the vessels of the body. As may be apparent, the disclosed infusion guidewire may be used for placement of a catheter within the vasculature of the human body, it may be used by itself to cross an obstruction within the vessels or it may be used to carry a therapeutic device mounted on the distal end of the infusion guidewire for purposes of removing obstructions which may exist within a vessel of the body.

The preceding specific embodiment is illustrated of the practice of this invention. It is to be understood, however, that other variations may also be employed without departing from the spirit and scope of the invention as hereinafter claimed.

That which is claimed is:

1. A bidirectional steerable infusion guidewire having a deflectable tip which comprises:
an elongated flexible tubing having proximal and distal ends;
a flexible helical coil having multiple turns and having proximal and distal ends, said helical coil being formed from an elongated member having a rectangular cross-sectional configuration and having continuous undulations wherein the undulations of adjacent turns interlock with each other in order to enhance the rotational rigidity of the coil, the proximal end of said helical coil is attached to the distal end of the flexible tubing;
an elongated deflection member having proximal and distal portions and being slidably disposed within said tubing and within said helical coil, the distal portion of said deflection member being flattened to form a deflection ribbon which extends in a plane;
a retaining ribbon having proximal and distal ends, the proximal end of the retaining ribbon is attached to the distal end of the flexible tubing and the retaining ribbon is oriented to extend in a plane which is generally parallel to the plane of the deflection ribbon;
an attachment member engaging the distal end of the helical coil, the distal portion of the deflection member and the distal end of the retaining ribbon so that longitudinal movement of the deflection member in a distal direction causes the distal end of the helical coil to be deflected in one direction and longitudinal movement of the deflection member in a proximal direction causes the distal end of the helical coil to deflect in another opposite direction; and,
an infusion tube extending through the elongated tubing from the proximal end of the elongated flexible tubing to the distal end of the elongated tubing and adapted to infuse a therapeutic agent.

2. A bi-directional steerable infusion guidewire as defined in claim 1, wherein said infusion tube extends through said attachment member to the distal end of said steerable infusion guidewire.

3. A bidirectional steerable infusion guidewire as defined in claim 1, wherein the continuous undulations take the form of a sinusoidal wave having positive and negative peaks and in which the positive peaks of adjacent turns of coils engage negative peaks of adjacent turns.

4. A bi-directional steerable infusion guidewire as defined in claim 2, wherein the continuous undulations take the form of a sinusoidal wave having positive and negative peaks and in which the positive peaks of adjacent turns of coils engage negative peaks of adjacent turns.

5. A bidirectional steerable infusion guidewire as defined in claim 1, wherein the continuous undulations take the form of a square sinusoidal wave having positive and negative peaks and in which the positive peaks of adjacent turns of the coil engage negative peaks of adjacent turns of the coil.

6. A bi-directional steerable infusion guidewire as defined in claim 2, wherein the continuous undulations take the form of a square sinusoidal wave having positive and negative peaks and in which the positive peaks of adjacent turns of the coil engage negative peaks of adjacent turns of the coil.

7. A bi-directional steerable infusion guidewire as defined in claim 1, wherein the deflection member has a square cross-sectional configuration.

8. A bi-directional steerable infusion guidewire as defined in claim 2, wherein the deflection member has a square cross-sectional configuration.

9. A bi-directional steerable infusion guidewire as defined in claim 1, wherein the retaining ribbon and the deflection member are normally biased in an arcuate configuration to thereby cause the distal end of the helical coil to be normally biased in a curved shape.

10. A bi-directional steerable infusion guidewire as defined in claim 2, wherein the retaining ribbon and the deflection member are normally biased in an arcuate configuration to thereby cause the distal end of the helical coil to be normally biased in a curved shape.

11. A bi-directional steerable infusion guidewire as defined in claim 1, wherein the proximal portion of said deflection member is of a circular cross section which extends from the proximal end of the flexible tubing to approximately the distal end of the elongated tubing.

12. A bi-directional steerable infusion guidewire as defined in claim 2, wherein the proximal portion of said deflection member is of a circular cross section which extends from the proximal end of the flexible tubing to approximately the distal end of the elongated tubing.

13. A bi-directional steerable infusion guidewire as defined in claim 12, wherein the proximal end of said retaining ribbon extends from the distal end of the flexible tubing to approximately the distal end of the flexible helical coil.

14. A bi-directional steerable infusion guidewire as defined in claim 11, wherein the proximal end of said retaining ribbon extends from the distal end of the flexible tubing to approximately the distal end of the flexible helical coil.

15. A bi-directional steerable infusion guidewire as defined in claim 1, wherein the attachment member takes the form of a rounded bead.

16. A bi-directional steerable infusion guidewire as defined in claim 2, wherein the attachment member takes the form of a rounded bead.

17. A bi-directional steerable infusion guidewire as defined in claim 15, wherein the rounded bead is formed with an epoxy material.

18. A bi-directional steerable infusion guidewire as defined in claim 16, wherein the rounded bead is formed with an epoxy material.

19. A bi-directional steerable infusion guidewire as defined in claim 1, wherein the attachment member takes the form of a rounded bead which contacts the distal end of the helical coil to define a circular surface at the distal end of the coil and the deflection ribbon engages the rounded bead at a location offset from the center of the circular surface of the rounded bead.

20. A bi-directional steerable infusion guidewire as defined in claim 2, wherein the attachment member takes the form of a rounded bead which contacts the distal end of the helical coil to define a circular surface at the distal end of the coil and the deflection ribbon engages the rounded bead at a location offset from the center of the circular surface of the rounded bead.

21. A bi-directional steerable infusion guidewire as defined in claim 19, wherein the distal end of the retaining ribbon engages the rounded bead at a location offset from the center of the circular surface of the rounded bead.

22. A bi-directional steerable infusion guidewire as defined in claim 20, wherein the distal end of the retaining ribbon engages the rounded bead at a location offset from the center of the circular surface of the rounded bead.

23. A bi-directional steerable infusion guidewire as defined in claim 21, wherein the distal end of the retaining ribbon engages the rounded bead at a location offset from the center of the circular surface in an opposite direction from the offset location of the deflection member.

24. A bi-directional steerable infusion guidewire as defined in claim 20, wherein the distal end of the retaining ribbon engages the rounded bead at a location offset from the center of the circular surface in an opposite direction from the offset location of the deflection member.

25. A bi-directional steerable infusion guidewire as defined in claim 24, wherein the deflection member and the retaining ribbon are joined to each other within the rounded bead.

26. A bi-directional steerable infusion guidewire as defined in claim 23, wherein the deflection member and the retaining ribbon are joined to each other within the rounded bead.

27. A steerable infusion guidewire having a deflectable tip which comprises:
an elongated flexible tubing having proximal and distal ends;
a flexible helical coil having multiple turns and having proximal and distal ends, said helical coil being formed from an elongated member having a rectangular cross-sectional configuration and having continuous undulations wherein the undulations of adjacent turns interlock with each other in order to enhance the rotational rigidity of the coil, the proximal end of said helical coil is attached to the distal end of the flexible tubing;
a rounded bead engaging the distal end of the helical coil; and,
an infusion tube extending through the elongated tubing from the proximal end of the elongated flexible tubing to the distal end of the elongated tubing and adapted to infuse a therapeutic agent.

28. A steerable infusion guidewire as defined in claim 27, wherein the continuous undulations take the form of a sinusoidal wave having positive and negative peaks and in which the positive peaks of adjacent turns of coils engage negative peaks of adjacent turns.

29. A steerable infusion guidewire as defined in claim 27, wherein the continuous undulations take the form of a square sinusoidal wave having positive and negative peaks and in which the positive peaks of adjacent turns of coils engage negative peaks of adjacent turns.

30. A steerable infusion guidewire as defined in claim 27, wherein the elongated member has a square cross-sectional configuration.
